(19) United States
(12) Patent Application Publication
(10) Pub. No.: US 2017/0273609 A1
(43) Pub. Date: Sep. 28, 2017

(54) METHOD AND APPARATUS FOR ASSESSING TISSUE OXYGENATION SATURATION

(71) Applicant: Indiana University Research and Technology Corporation, Indianapolis, IN (US)

(72) Inventors: Luke G. Gutwein, Indianapolis, IN (US); Clinton D. Bahler, Indianapolis, IN (US); Anthony S. Kaleth, Zionsville, IN (US)

(73) Assignee: Indiana University Research and Technology Corporation, Indianapolis, IN (US)

(21) Appl. No.: 15/466,751

(22) Filed: Mar. 22, 2017

Related U.S. Application Data
(60) Provisional application No. 62/411,160, filed on Oct. 21, 2016.
(60) Provisional application No. 62/313,552, filed on Mar. 25, 2016.

(51) Int. Cl. A61B 5/1455; A61B 5/0205; A61B 5/00

(52) U.S. Cl. CPC A61B 5/14552; A61B 5/6807; A61B 5/02055; A61B 5/02438

(57) ABSTRACT

A method and apparatus for assessing tissue oxygenation saturation during physical activity comprising the steps of providing at least one near-infrared spectroscopy probe including at least one near-infrared light source and at least one photodetector-and measuring oxygen saturation in at least one of a skin dermis layer, an adipose layer and a muscular fascial layer of a user.

### RELATED APPLICATIONS

This application claims priority to U.S. Provisional Patent Application Ser. No. 62/411,160, filed Oct. 21, 2016, titled “Portable Near-Infrared Spectroscopy Apparatus,” to Gutwein et al. and U.S. Provisional Patent Application Ser. No. 62/313,552, filed Mar. 25, 2016, titled “Method and Apparatus for Assessing Tissue Oxygenation Saturation,” to Gutwein et al., the entire disclosures of which are expressly incorporated by reference herein.

### FIELD OF DISCLOSURE

The present disclosure relates generally to a method and apparatus for assessing tissue oxygenation saturation using near-infrared spectroscopy, more particularly, a method and apparatus for assessing tissue oxygenation saturation in skin, adipose and muscular fascial tissues in real-time during physical activity.

### BACKGROUND OF DISCLOSURE

Near-infrared spectroscopy (NIRS) is a well-established optical technique that allows for continuous non-invasive monitoring of hemoglobin oxygen saturation in microvessels. Near-infrared light (600-1000 nm) can penetrate biologic tissue 2-6 centimeters and is either absorbed or scattered within the tissue. The ability to measure tissue oxygenation with near-infrared light was principally founded on the Beer-Lambert Law, and exists due to the differential light absorption coefficients of oxyhemoglobin and deoxyhemoglobin. This technique uses the absorption characteristics of near-infrared light to calculate changes in oxygenated and deoxygenated hemoglobin in muscle tissue.

Today, medical and optical application of near-infrared light is prolific. Among the applications used are the study of septic shock, free tissue transfer, real-time tissue perfusion analysis during surgery, cancer nanotechnology, and peripheral arterial disease. In addition to its numerous clinical applications, NIRS has been widely used in sports and exercise physiology research to monitor local oxygenation adaptations in skeletal muscle during exercise. Previous studies have shown NIRS to be highly sensitive to changes in muscle tissue oxygenation (StO₂) during exercise. The NIRS signal obtained during exercise is considered to reflect the balance between oxygen delivery and utilization, evidenced by the gradual decrease in skeletal muscle oxygenation during incremental exercise and by the decrease in oxygenation that is proportional to the intensity during steady-state exercise.

To date, no standardization is available for NIRS instrumentation. However, due to their relatively low cost and ease of use, the majority of commercially available NIRS devices are continuous wave (CW) spectrometers. As it pertains to the study of human locomotion, an important aspect of any NIRS device is transportability, which allows for the study of skeletal muscle oxygenation during exercise in a field setting. The majority of early NIRS devices could only be used for laboratory-based research, as they required a physical connection between the person, an external power supply, and the data storage center. In recent years, commercial manufacturers have been developing smaller NIRS systems with wireless probes attached to the muscles of interest with adhesive tape.

This advance in technology has enabled researchers to continuously monitor tissue oxygenation changes during various exercises or sport-specific activities outside the laboratory environment. However, despite these steps forward in NIRS technology, its usefulness in monitoring muscle oxygenation during dynamic exercise remains clouded for several reasons, including device size, motion signal artifact, and the influence of adipose tissue thickness on probe penetration depth.

The path of light through tissue follows a banana-shaped curve and the depth is determined by the distance between the emitting source and a detector of near-infrared light. The larger the source-detector distance the deeper the penetration of a NIRS probe. Penetration depth is expected to be approximately half the distance of the source-detector distance. Generally, the depth sensitivity is accepted to be 0.43 times the source-detector distance. However, since the depth of penetration is resultant of the path of light through the tissue, NIRS probes are generally of substantial size. PortaMon (Artinis Medical Systems B.V. Netherlands) is the most commonly used portable NIRS device and measures 83.8 millimeters by 42.9 millimeters by 17.2 millimeters. Present NIRS devices for exercise application have an approximately 15 millimeter depth sensitivity based on a source-detector distance of 30-40 millimeters. For the PortaMon device, source-detector distances are 30 millimeters, 35 millimeters, and 40 millimeters between a single receiver and three transmitters.

NIRS technology also is limited by signal motion artifact due to instability at the probe-skin interface during dynamic exercise. Therefore, the reliability of measurement has been questioned, and consequently, many prior studies have been limited to low-impact exercises such as a cycling ergometer or isometric contraction.

Additionally, the influence of skin and adipose tissue on the measurement of StO₂ during exercise has been questioned. However, skin blood flow during dynamic exercise has been found to not impact the StO₂ measurement. In a study, skin was heated to 37 and 40 degrees to obtain vasodilation at rest as well as during knee extension exercises. A slight increase in StO₂ at rest was observed but no change was observed during exercise. The source-detector distance in this study was 50 mm. Nevertheless, the significance of the omnipresent adipose layer has been debated and thought to impede the accurate assessment of muscle oxygenation if the adipose layer thickness is beyond 5 mm for a 40 mm source-detector distance.

Moreover, the microcirculation present in the pen-muscular environment of the fascia, adipose tissue, and subdermal plexus has not been exclusively studied with NIRS during exercise. Anatomy of the skin, adipose, and fascial layer proves that the arterial supply and venous drainage originate from perforating vessels from the local muscle. Recently, the perforasome theory was introduced, which described how blood supply to the vascular territory of the skin and subcutaneous adipose tissue originate from penetrating or perforating vessels (perforasomes) of the deep muscular fascia or muscular fascial septum. Evidence to support this theory is demonstrated by the ability to move skin-adipose tissue flap to another area of the body with or without an arterial and venous microsurgical anastomosis. Common skin adipose flaps include the deep inferior epigastric perforator flap for breast reconstruction and the anterolateral thigh flap to aid wound closure after cancer extirpation. Therefore, the microcirculatory milieu (or perforasomes) of skin and adipose tissue may be a direct window of local exercising muscle oxygenation kinetics and metabolic demand of the muscle.

Accordingly, a method and apparatus for assessing tissue oxygenation saturation within a skin dermis layer, an adipose layer or a muscular fascial layer is still needed, more particularly, a method and apparatus for measuring tissue oxygenation saturation within skin, adipose or muscular fascial layers in real-time during physical activity.

### SUMMARY OF THE DISCLOSURE

In one embodiment of the present disclosure, a portable near-infrared spectroscopy apparatus is provided. The apparatus generally includes at least one wearable article and at least one near-infrared spectroscopy probe coupled to the at least one wearable article, the at least one near-infrared spectroscopy probe including at least one near-infrared light source and at least one photodetector, wherein the at least one near-infrared spectroscopy probe is configured to measure oxygenation saturation of at least one of a skin dermis layer, an adipose layer and a muscular fascial layer.

In one aspect of the apparatus, the at least one near-infrared light source and the at least one photodetector are approximately 1-15 millimeters apart.

In another aspect of the apparatus, the at least one near-infrared light source emits light having wavelengths between 600 and 1000 nanometers.

In a further aspect of the apparatus, the at least one near-infrared spectroscopy probe includes two near-infrared light sources, one of the light sources emitting light with a wavelength of 690 nanometers and the other light source emitting light with a wavelength of 830 nanometers.

In another aspect of the apparatus, the at least one near-infrared spectroscopy probe emits light with a scan rate of up to 40 Hertz.

In another aspect of the apparatus, the at least one wearable article is a one-piece garment.

In another aspect of the apparatus, the at least one wearable article is at least one of a top, a bottom and at least one calf stocking.

In a further aspect of the apparatus, the at least one wearable article includes at least one integrated compartment, the at least one near-infrared spectroscopy probe being stored within the at least one integrated compartment.

In another aspect of the apparatus, the at least one near-infrared spectroscopy probe is a fiber optic probe.

In a further aspect of the apparatus, the at least one near-infrared spectroscopy probe includes two near-infrared light sources and four photodetectors.

In another aspect of the apparatus, the at least one near-infrared spectroscopy probe has a depth sensitivity of approximately 2.3 to 3 millimeters.

In another aspect of the apparatus, the at least one near-infrared spectroscopy probe further includes a microprocessor configured to operate the at least one near-infrared light source and the at least one photodetector.

In another aspect of the apparatus, the at least one near-infrared spectroscopy probe includes two sides with a length of approximately 8 millimeters, two sides with a length of approximately 12 millimeters and a depth of approximately 4 millimeters.

In another embodiment of the present disclosure, a method for assessing tissue oxygenation saturation during physical activity is provided. The method includes providing at least one near-infrared spectroscopy probe including at least one near-infrared light source and at least one photodetector, and measuring oxygen saturation in at least one of a skin dermis layer, an adipose layer and a muscular facial layer of a user.

In one aspect of the method, the at least one near-infrared spectroscopy probe is an oximeter.

In another aspect of the method, the near-infrared light source and the detector are approximately 1-15 millimeters apart.

In a further aspect of the method, the step of measuring oxygen saturation in at least one of a skin dermis layer and an adipose layer includes collecting measurements every six seconds.

In another aspect of the method, the method further includes measuring at least one of a heart rate, a respiratory rate and a body temperature of the user.

In another aspect of the method, the at least one near-infrared spectroscopy probe further includes a transmitter.

In a further aspect of the method, the method further includes transmitting data from the at least one near-infrared spectroscopy probe to an external display device via one of a hardwire connection and a wireless connection.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWING

The foregoing aspects and many of the intended advantages of this disclosure will become more readily appreciated as the same becomes better understood by reference to the following detailed description when taken in conjunction with the accompanying drawing.

FIG. 1 is a view of a user wearing a portable near-infrared spectroscopy apparatus of the present disclosure;

FIG. 2A is a graph demonstrating data compiled from a study of the medial gastrocnemius (MG) and the vastus medialis (VM) using an embodiment of a probe of the present disclosure during running exercises;

FIG. 2B is a graph demonstrating data compiled from a study of the medial gastrocnemius (MG) and the vastus medialis (VM) an embodiment of a portable near-infrared spectroscopy apparatus of the present disclosure during running exercises with an increasing grade and fixed running rate;

FIG. 2C is a graph demonstrating data compiled from a study of the medial gastrocnemius (MG) and the vastus medialis (VM) an embodiment of a portable near-infrared spectroscopy apparatus of the present disclosure during running exercises with an increasing running rate and a fixed grade;

FIG. 3A is a graph demonstrating data compiled from a study of the medial gastrocnemius (MG) using an embodiment of a portable near-infrared spectroscopy apparatus of the present disclosure during weight lifting exercises; and

FIG. 3B is a graph demonstrating data compiled from a study of the biceps brachii (BB) using an embodiment of a portable near-infrared spectroscopy apparatus of the present disclosure during weight lifting exercises.

Although the drawing represents an embodiment of various features and components according to the present disclosure, the drawing is not necessarily to scale and certain features may be exaggerated in order to better illustrate and explain the present disclosure. The exemplification set out herein illustrates embodiments of the disclosure, and such exemplifications are not to be construed as limiting the scope of the disclosure in any manner.

### DETAILED DESCRIPTION

For the purposes of promoting an understanding of the principals of the disclosure, reference will now be made to the embodiment illustrated in the drawing, which is described below. The embodiments disclosed below are not intended to be exhaustive or limit the disclosure to the precise form disclosed in the following detailed description. Rather, the embodiments are chosen and described so that others skilled in the art may utilize their teachings. It will be understood that no limitation of the scope of the disclosure is thereby intended. The disclosure includes any alterations and further modifications in the illustrative devices and described methods and further applications of the principles of the disclosure which would normally occur to one skilled in the art to which the disclosure relates.

A method and portable near-infrared spectroscopy apparatus are disclosed for measuring tissue oxygenation saturation during any type of exercise, sporting activity or activity of physical exertion. For instance, the disclosed method and/or apparatus may be used while a user 1 is walking, running, bicycling, swimming, cross training, or weight lifting.

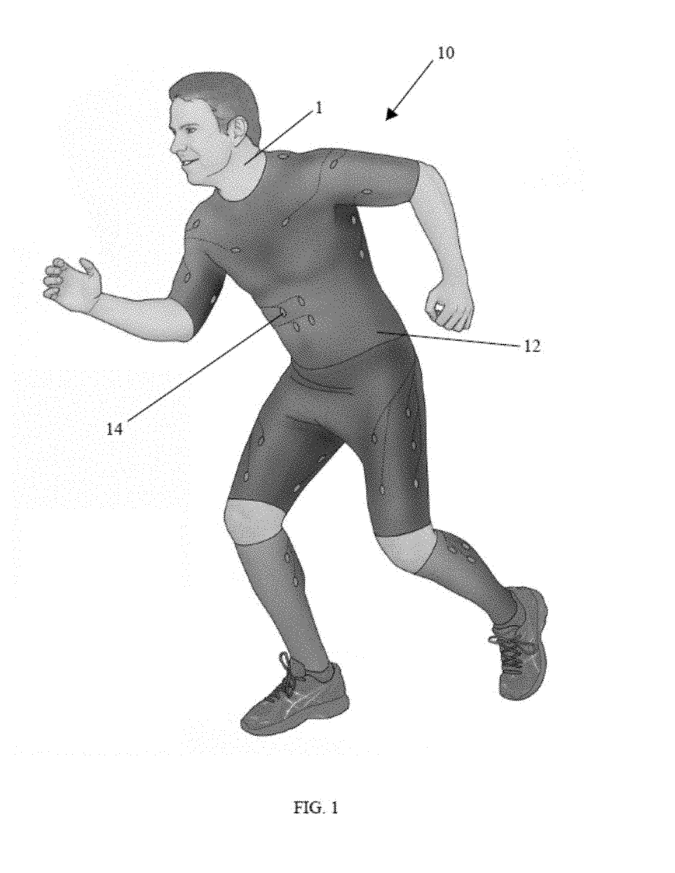

Referring to FIG. 1, a portable near-infrared spectroscopy apparatus 10 generally includes at least one wearable article 12 and at least one near-infrared spectroscopy probe 14 integrated into wearable article 12. In various embodiments, apparatus 10 may include multiple probes 14. Probe(s) 14 may further include an attachment cable (not shown), both of which may be integrated into apparatus 10 between layers of material that compose wearable article(s) 12 of apparatus 10. Additionally, probe 14 may be integrated into wearable article 12 such that optimal probe-skin apposition is provided. Further, wearable article 12 may include a transparent window, wherein probe 14 may interface directly with the skin or the transparent window of wearable article 12.

Still referring to FIG. 1, wearable article 12 may be a one-piece garment, a single article or a combination of multiple articles. For instance, wearable article 12 may be a top, a bottom and/or at least one calf stocking. In an exemplary embodiment, wearable article 12 is a one-piece garment covering a user's torso and at least a portion of the user's arms and legs. Additionally, wearable article 12 may be fitted or compressive to the body of user 1. In an exemplary embodiment, wearable article 12 may be made of elastic textile fibers. Furthermore, wearable article 12 may also include an integrated compartment wherein a probe 14, a light source and/or a detector may be stored.

In more detail, and still referring to FIG. 1, probe 14 may be a fiber optic probe. Generally, probe 14 includes at least one light source and at least one photodetector. In an exemplary embodiment, probe 14 includes two light sources and four photodetectors. Furthermore, in various embodiments, probe 14 may be an oximeter. Additionally, in various embodiments, the at least one light source and the at least one photodetector may be less than or equal to 15 millimeters apart, while in other various embodiments, the at least one light source and the at least one photodetector may be more than 15 millimeters apart. In an exemplary embodiment, the at least one light source and the at least one photodetector may be approximately 6 millimeters apart with a depth sensitivity of approximately 2.3-3 millimeters. Probe 14 may also include a microprocessor for operating the at least one light source and the at least one photodetector. Further, probe 14 may also include a casing. The measurements of probe 14 may be less than 83 millimeters by 42 millimeters by 17 millimeters. In an exemplary embodiment, probe 14, with or without a casing, may measure approximately 8 millimeters by 12 millimeters by 4 millimeters.

Moreover, the at least one light source may emit near-infrared light of wavelengths in the range of 600-1000 nanometers. In an exemplary embodiment, the at least one light source may be an oximeter. In an exemplary embodiment, the probe may include two light sources emitting 690 nanometer and 830 nanometer wavelengths at a scan rate of up to 40 Hertz via a fiberglass cable. Accordingly, measurements may be collected at any time interval. In various embodiments, measurements are collected every six seconds during baseline (pre-exercise), exercise and recovery (post-exercise).

Beyond this, probe 14 may be used to measure vital signs or oxygenation kinetics of muscle groups in user 1. In various embodiments, probe 14 may measure heart rate, respiratory rate, and/or body temperature. Additionally, probe 14 may be used to monitor electrical activity of the heart and/or pulse oximetry. More particularly, probe 14 may be used to measure the oxygenation kinetics of specific muscle group(s), for instance those within the pen-muscular microcirculatory environment. In various embodiments, the specified muscle groups may include at least one of trapezius, deltoid, biceps brachii, triceps, pectoralis major, latissimus dorsi, rectus abdominus, vastus medialis, vastus lateralis, biceps femoris, medical gastrocnemius, and lateral gastrocnemius. In an exemplary embodiment, the tissue oxygenation saturation (StO2) may be measured in the pen-muscular microcirculatory environment, and probe 14 may automatically generate tissue oxygenation saturation data for each measured individual muscle group. In one embodiment, probe 14 may be used to measure the oxygenation saturation of a skin dermis layer, an adipose layer and/or a muscular fascial layer in the pen-muscular microcirculatory environment.

For each muscle, probe 14 is generally positioned in the same location as determined by measurement for anatomical landmarks (i.e. tibial tuberosity). Each site is identified in the center of the muscle belly where signal quality of the tissue oximeter is greater than 80%. At probe location sites, depth from the skin surface to the muscle-fascial interface at least for the medial gastrocnemius and the vastus medialias is approximately 7.5 millimeters and 7.6 millimeters, respectively.

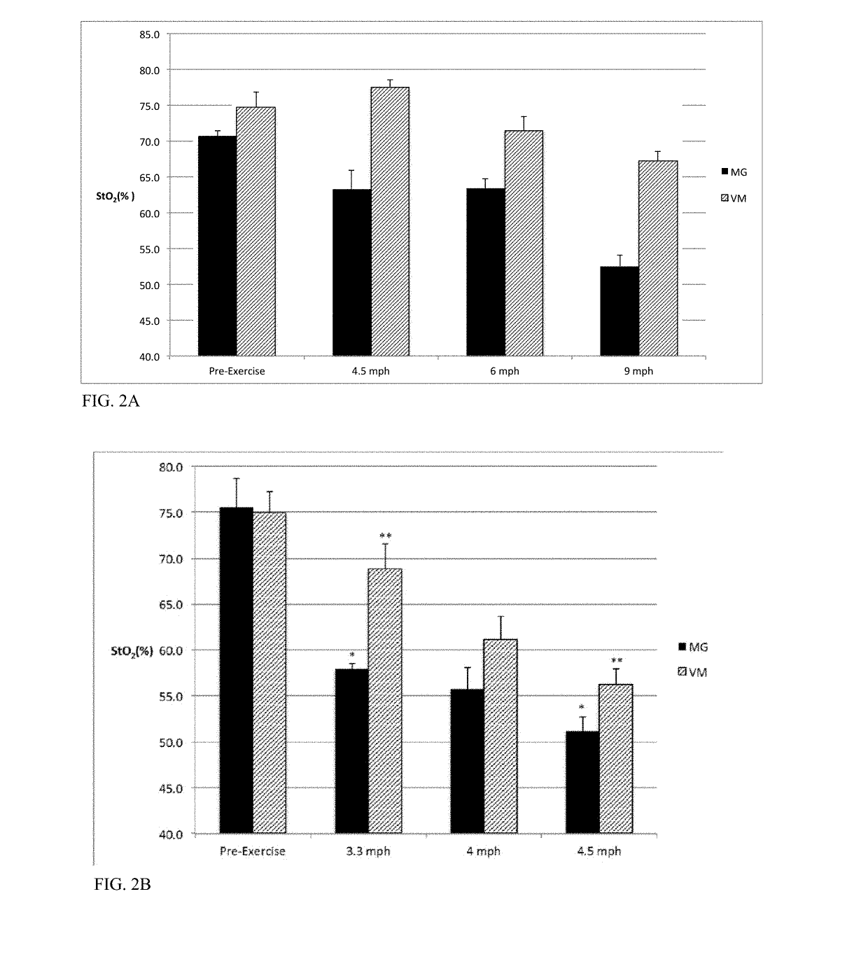

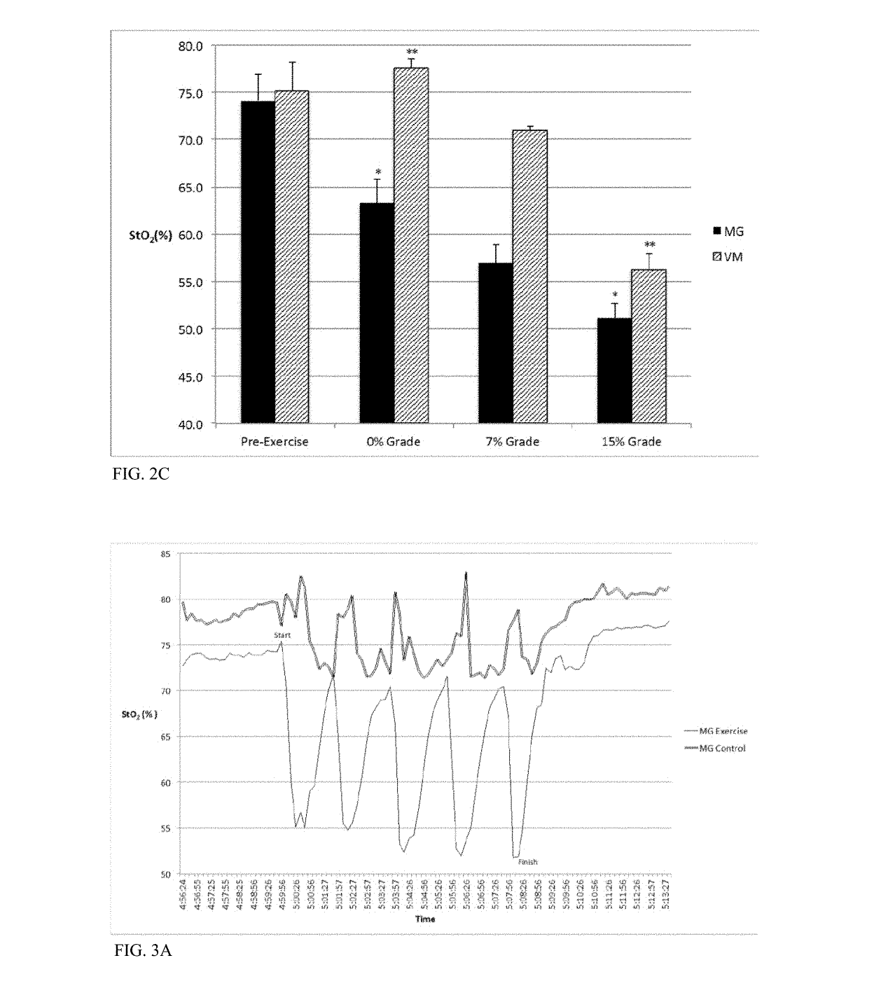

With reference to FIGS. 2A-C, graphs are shown demonstrating data compiled from a study of the medial gastrocnemius (MG) and the vastus medialis (VM) using an embodiment of probe 14 during running exercises with increased speed (FIG. 2A) and using an embodiment of apparatus 10 during running exercises (FIG. 2B-C). Example data recovered demonstrated that with increasing exercise intensity, a predictable and significant increase in oxygen extraction occurs in the pen-muscular environment of the MG and VM, and StO₂ decreases in the MC and VM. FIGS. 2B and 2C demonstrate this decrease of the mean StO₂ in the medial gastrocnemius and vastus medialis with an increasing grade and fixed running rate (FIG. 2C) or an increasing running rate and a fixed grade of 15% (FIG. 2B). This data demonstrates that when the user was running at a speed of 3.3 miles per hour at a 15% grade, the mean StO₂ of the medial gastrocnemius was 58%, while when the user was running at a speed of 4.5 at a 15% grade, the mean StO₂ dropped to 51.2% (FIG. 2B). Additionally, when the user was running at a speed of 4.5 miles per hour at a 0% grade, the mean StO₂ of the medial gastrocnemius was 63.2%, while when the user was running at a speed of 4.5 miles per hour at a 15% grade, the mean StO₂ dropped to 51.2% (FIG. 2C).

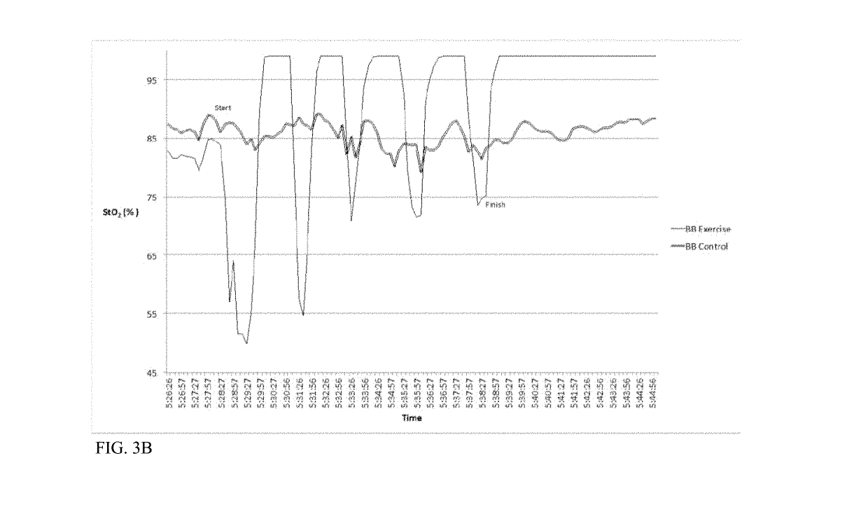

With reference to FIGS. 3A-B, graphs are shown demonstrating data compiled from a study of the medial gastrocnemius and the biceps brachii (BB) using an embodiment of apparatus 10 during weight lifting exercises. Furthermore, the graphs in FIGS. 3A and 3B demonstrate the StO₂ percentage of the medial gastrocnemius (FIG. 3A) and the biceps brachii (BB) (FIG. 3B) during weight lifting exercises. Not only does the exerted muscle group's StO₂ percentage decrease more compared to the non-exerted muscle group control, but a lesser decrease in StO₂ in the medial gastrocnemius or the biceps brachii can signify improper form being used. For instance, the last four repetitions in FIG. 3B demonstrate less of a decrease in StO₂ percentage as compared to the first repetition. Thus, it is likely that muscle fatigue caused the user to exercise poor athletic form wherein accessory muscles were being used instead of the target muscle. Thus, the measurements of StO₂ may allow the user to have a more effective workout by ensuring that proper form and muscles are being used, and the similar intensity is being used by measuring the StO₂ feedback.

Furthermore, in various embodiments, probe 14 may be in connection with a transmitter or other device such that data from probe 14 may be communicated to an external display or device (i.e., smart phone) via a hardwire connection or a wireless connection. In an exemplary embodiment, the connection is wireless. Additionally, the display or device receiving the probe data may also contain an algorithm such that the display or device may show data received, store data received, provide an intensity rating based on data received and/or compare previous data to current data. Furthermore, probe 14 may also include a memory chip for storing data.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A portable near-infrared spectroscopy apparatus comprising:
at least one wearable article; and
at least one near-infrared spectroscopy probe coupled to the at least one wearable article, the at least one near-infrared spectroscopy probe including at least one near-infrared light source and at least one photodetector, wherein the at least one near-infrared spectroscopy probe is configured to measure oxygenation saturation of at least one of a skin dermis layer, an adipose layer and a muscular fascial layer of a user during physical activity.

2. The apparatus of claim 1, wherein the at least one near-infrared light source and the at least one photodetector are approximately 1-15 millimeters apart.

3. The apparatus of claim 1, wherein the at least one near-infrared light source emits light having wavelengths between 600 and 1000 nanometers.

4. The apparatus of claim 3, wherein the at least one near-infrared spectroscopy probe includes two near-infrared light sources, one of the light sources emitting light with a wavelength of 690 nanometers and the other light source emitting light with a wavelength of 830 nanometers.

5. The apparatus of claim 1, wherein the at least one near-infrared spectroscopy probe emits light with a scan rate of up to 40 Hertz.

6. The apparatus of claim 1, wherein the at least one wearable article is a one-piece garment.

7. The apparatus of claim 1, wherein the at least one wearable article is at least one of a top, a bottom and at least one calf stocking.

8. The apparatus of claim 1, wherein the at least one wearable article includes at least one integrated compartment, the at least one near-infrared spectroscopy probe being stored within the at least one integrated compartment.

9. The apparatus of claim 1, wherein the at least one near-infrared spectroscopy probe is a fiber optic probe.

10. The apparatus of claim 1, wherein the at least one near-infrared spectroscopy probe includes two near-infrared light sources and four photodetectors.

11. The apparatus of claim 1, wherein the at least one near-infrared spectroscopy probe has a depth sensitivity of approximately 2.3 to 3 millimeters.

12. The apparatus of claim 1, wherein the at least one near-infrared spectroscopy probe further includes a microprocessor configured to operate the at least one near-infrared light source and the at least one photodetector.

13. The apparatus of claim 1, wherein the at least one near-infrared spectroscopy probe includes two sides with a length of approximately 8 millimeters, two sides with a length of approximately 12 millimeters and a depth of approximately 4 millimeters.

14. A method for assessing tissue oxygenation saturation during physical activity comprising the steps of:
positioning at least one near-infrared spectroscopy probe in close proximity to skin of a user, the at least one near-infrared spectroscopy probe including at least one near-infrared light source and at least one photodetector;
measuring, with the at least one near-infrared spectroscopy probe, oxygen saturation in at least one of a skin dermis layer, an adipose layer and a muscular fascial layer of the user during the physical activity.

15. The method of claim 14, wherein the at least one near-infrared spectroscopy probe is an oximeter.

16. The method of claim 14, wherein the near-infrared light source and the detector are approximately 1-15 millimeters apart.

17. The method of claim 14, wherein the step of measuring oxygen saturation in at least one of a skin dermis layer and an adipose layer includes collecting measurements every six seconds.

18. The method of claim 14 further comprising:
measuring at least one of a heart rate, a respiratory rate and a body temperature of the user.

19. The method of claim 14, wherein the at least one near-infrared spectroscopy probe further includes a transmitter.

20. The method of claim 19 further comprising:
transmitting data from the at least one near-infrared spectroscopy probe to an external display device via one of a hardwire connection and a wireless connection.
